(19) United States
(12) Patent Application Publication
(10) Pub. No.: US 2008/0140101 A1
(43) Pub. Date: Jun. 12, 2008

(54) APPARATUS FOR CROSSING OCCLUSIONS OR STENOSES

(75) Inventors: Michael Carley, San Jose, CA (US); Victor Chechelski, Mountain View, CA (US); Rudolfo Sudaria, San Jose, CA (US); Nestor Aganon, San Jose, CA (US); Christopher Huynh, San Jose, CA (US); Gerardo V. Noriega, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ReVascular Therapeutic, Inc., Sunnyvale, CA (US)

(21) Appl. No.: 11/567,884

(22) Filed: Dec. 7, 2006

(51) Int. Cl. A61B 17/22

(52) U.S. Cl. 606/159

(57) ABSTRACT

A torqueable hollow device, such as a hollow guidewire device, with a pre-determined fixed distal tip is disclosed for removing occlusive material and passing through occlusions, stenosis, thrombus, plaque, calcified material, and other materials in a body lumen, such as a coronary artery. The hollow guidewire generally comprises an elongate, tubular guidewire body that has an axial lumen. A mechanically moving core element is positioned at or near a distal end of the tubular guidewire body and extends through the axial lumen. Actuation of the core element (e.g., oscillation, reciprocation, and/or rotation) creates a passage through the occlusive or stenotic material in the body lumen.

### CROSS-REFERENCES TO RELATED APPLICATIONS

The present application is related to U.S. patent application Ser. No. 09/030,657, Attorney Docket No. 019635-000100US, filed Feb. 25, 1998, entitled “Steerable Unitary Infusion Catheter/Guide Wire Incorporating Detachable Infusion Port Assembly,” and now U.S. Pat. No. 6,059,767, and U.S. patent application Ser. No. 09/935,534, Attorney Docket No. 019635-000310US, filed Aug. 22, 2001, entitled “Steerable Support System with External Ribs/Slots that Taper,” and now U.S. Pat. No. 6,746,422, the complete disclosures of which are incorporated herein by reference, in their entirety. The present application is also related to U.S. patent application Ser. No. 11/236,703, Attorney Docket No. 019635-000240US, filed Sep. 26, 2005, entitled “Guidewire for Crossing Occlusions or Stenoses,” which was a continuation-in-part of U.S. patent application Ser. No. 10/999,457, Attorney Docket No. 019635-000500US, filed Nov. 29, 2004, entitled “Guidewire For Crossing Occlusions or Stenoses,” which was a continuation-in-part of U.S. patent application Ser. No. 09/644,201, Attorney Docket No. 019635-000210US, filed Aug. 22, 2000, entitled “Guidewire for Crossing Occlusions or Stenoses,” and now U.S. Pat. No. 6,824,550, which claimed the benefit under 37 C.F.R. § 1.78 to U.S. Provisional Patent Application No. 60/195,154, Attorney Docket No. 019635-000200US, filed Apr. 6, 2000, entitled “Guidewire for Crossing Occlusions or Stenosis,” and U.S. patent application Ser. No. 11/388,251, Attorney Docket No. 019635-001200US, filed Mar. 22, 2006, entitled “Guidewire Controller System,” the complete disclosures of which are incorporated herein by reference, in their entirety.

### BACKGROUND OF THE INVENTION

The present invention is generally related to medical devices, kits, and methods. More specifically, the present invention provides a guidewire system for crossing stenosis, partial occlusions, or total occlusions in a patient's body.

Cardiovascular disease frequently arises from the accumulation of atheromatous material on the inner walls of vascular lumens, particularly arterial lumens of the coronary and other vasculature, resulting in a condition known as atherosclerosis. Atheromatous and other vascular deposits restrict blood flow and can cause ischemia which, in acute cases, can result in myocardial infarction or a heart attack. Atheromatous deposits can have widely varying properties, with some deposits being relatively soft and others being fibrous and/or calcified. In the latter case, the deposits are frequently referred to as plaque. Atherosclerosis occurs naturally as a result of aging, but may also be aggravated by factors such as diet, hypertension, heredity, vascular injury, and the like.

Atherosclerosis can be treated in a variety of ways, including drugs, bypass surgery, and a variety of catheter-based approaches which rely on intravascular widening or removal of the atheromatous or other material occluding the blood vessel. Particular catheter-based interventions include angioplasty, atherectomy, laser ablation, stenting, and the like. For the most part, the catheters used for these interventions must be introduced over a guidewire, and the guidewire must be placed across the lesion prior to catheter placement. Initial guidewire placement, however, can be difficult or impossible in tortuous regions of the vasculature. Moreover, it can be equally difficult if the lesion is total or near total, i.e. the lesion occludes the blood vessel lumen to such an extent that the guidewire cannot be advanced across the lesion.

To overcome this difficulty, forward-cutting atherectomy catheters have been proposed. Such catheters usually can have a forwardly disposed blade (U.S. Pat. No. 4,926,858) or rotating burr (U.S. Pat. No. 4,445,509). While effective in some cases, these catheter systems, even when being advanced through the body lumen with a separate guidewire, have great difficulty in traversing through the small and tortuous body lumens of the patients and reaching the target site.

For these reasons, it is desired to provide devices, kits, and methods which can access small, tortuous regions of the vasculature and which can remove atheromatous, thrombotic, and other occluding materials from within blood vessels. In particular, it is desired to provide atherectomy systems which can pass through partial occlusions, total occlusions, stenosis, and be able to macerate blood clots or thrombotic material. It is further desirable that the atherectomy system have the ability to infuse and aspirate fluids before, during, or after crossing the lesion. At least some of these needs will be met by the devices and methods of the present invention described hereinafter and in the claims.

### BRIEF SUMMARY OF THE INVENTION

The systems, devices and methods according to the present invention will generally be adapted for the intraluminal treatment of a target site within a body lumen of a patient, usually in a coronary artery or peripheral blood vessel which is occluded or stenosed with atherosclerotic, stenotic, thrombotic, or other occlusive material. The systems, devices and methods, however, are also suitable for treating stenoses of the body lumens and other hyperplastic and neoplastic conditions in other body lumens, such as the ureter, the biliary duct, respiratory passages, the pancreatic duct, the lymphatic duct, and the like. Neoplastic cell growth will often occur as a result of a tumor surrounding and intruding into a body lumen. Removal of such material can thus be beneficial to maintain patency of the body lumen. While the remaining discussion is directed at passing through atheromatous or thrombotic occlusive material in a coronary artery, it will be appreciated that the systems and methods of the present invention can be used to remove and/or pass through a variety of occlusive, stenotic, or hyperplastic material in a variety of body lumens. It should also be appreciated, that many of the features of the different embodiments as described, may be used in the described embodiment or together with others. More particularly, the present invention can be used for passing through stenosis or occlusions in a neuro, cardio, and peripheral body lumens. Generally, the present invention includes an elongate member, such as a hollow body such as hollow body guidewire, that is advanced through a body lumen and positioned adjacent the occlusion or stenosis. The guidewire body may include a hub at a proximal end to ease receiving over or being received over other elongate members such as access systems including therapeutic (e.g., balloon catheter) or catheters used for accessing target site. The hollow devices of the present invention, unless otherwise stated, may generally have similar dimensions as those of conventional guidewires. Devices of the present invention, such as hollow guidewire devices, may be used alone or in combination with other elongate members such as conventional guidewires and access systems.

In an embodiment, devices of the present invention include a hollow body, such as a hollow guidewire body, having a pre-determined fixed deflected distal end, as compared to a longitudinal axis of the hollow guidewire (i.e., a deflection angle as defined by a tangential line formed between the distal end of the guidewire body and its longitudinal axis).

An occlusive material (e.g., plaque) removal assembly is positioned at or near a distal tip of the hollow guidewire to create an opening in the occlusion. In an embodiment, the plaque removal assembly comprises a core element having a drive shaft and a distal tip that is configured for oscillation, reciprocation (e.g., pecking), and/or rotation and disposed within the axial lumen of the hollow guidewire and extending distally from the guidewire distal end. In an embodiment, the distal tip of the core element may be configured for further advancement and/or retraction from the distal end of the hollow guidewire. Once the guidewire has reached the lesion, the guidewire with the exposed drive shaft may be advanced into the lesion. Alternatively, the guidewire may be disposed in a relatively fixed position, and the drive shaft may be advanced to create an opening forward of the hollow guidewire forming a path in the occlusion or stenosis. In an embodiment, the core element is configured for rotational oscillation.

By way of example, and not limitation, it was found that while a deflection angle is advantageous to allow the user to torque the guidewire to re-direct the tip, as the deflection angle increases the axial penetration force decreases. The pre-determined fixed deflection of the distal end of the guidewire body according to the present invention, ranges from about 0° to about 90 degrees (“°”), from about 0° to about 60°, from about 5° to about 45°. In an embodiment, the pre-determined fixed deflection is about 15°, about 30°, or about 45°. The fixed deflection of the distal end of the hollow guidewire body may be arrived at in a smooth transition or in an abrupt transition, or any type and degree of transition inbetween. To facilitate passing through the occlusion or stenosis, the distal end of the hollow guidewire can be steerable to provide better control of the creation of the path through the occlusion or stenosis. Optionally, the target site can be infused and/or aspirated before, during, and after creation of the path through the occlusion.

By way of example, in an embodiment, a 15° fixed angle of deflection may be advantageous for re-directing the tip while still maintaining substantial axial penetration force. Alternatively, in another embodiment, a smaller deflection angle may be required to increase penetration force or allow for better alignment in straight lesions. Alternatively, in another embodiment, a larger deflection angle may be required in tortuous anatomies. Crossing an occlusion may require the use of two or more fixed deflection guidewires according to the present invention, each having a different fixed angle of deflection based on the characteristics of different segments of the lesion for treatment of which it is used.

In an embodiment, the pre-determined fixed deflection is, at least in part, achieved by way of an elongate body such as a metal wire or ribbon longitudinally disposed within the distal portion of the hollow guidewire inner lumen and is fixedly attached to an inner surface thereof. The elongate body may have a flat or arcuate (e.g., crescent shape) transverse profile. In an embodiment, the metal wire or ribbon is attached to the inner lumen along at least a distal attachment point at the hollow guidewire distal end 22 and at a proximal attachment point proximally extending from the hollow guidewire distal end. In an embodiment, the elongate body conforms to the inner diameter of the distal portion of the hollow guidewire when it is attached thereto by suitable means, such as soldering. The metal wire or ribbon (with flat or curved profile) may be formed from suitable material such as stainless steel, nitinol, or cobalt-chromium; and has a longitudinal dimension ranging from about 0.3 centimeters (“cm”) to about 6 cm, from about 0.5 cm to about 2 cm. In an embodiment, the metal wire or ribbon 50 has a longitudinal dimension of about 1 cm.

In an embodiment, the pre-determined fixed deflection is, at least in part, achieved by way of a shaped distal portion of the guidewire body. For example, the shaped distal portion may be made from a nickel-titanium alloy and heat set to the pre-determined fixed deflection angle. In such an embodiment, the distal portion may, optionally, also include the elongate body such as the metal wire or ribbon as further means to provide the pre-determined fixed deflection.

The hollow guidewire of the present invention has a pre-determined distal deflection, flexibility, pushability, and torqueability to be advanced through the tortuous blood vessel without the use of a separate guidewire or other guiding element. Additionally, the hollow guidewire may be sized to fit within an axial lumen of a conventional support or access catheter system. The distal end of the hollow guidewire, in relaxed unconstrained state, has a pre-determined angle of deflection. The distal end deflection is designed such that when the guidewire is housed within and introduced through another elongate body, such as a balloon catheter, the angle of the deflected distal end of the guidewire may at least be partially decreased (e.g., straightened) to accommodate the inner diameter of the catheter. Once the guidewire exits the catheter (e.g., balloon catheter), the distal end returns to its preset deflected angle.

The catheter system can be delivered either concurrently with the advancement of the hollow guidewire or after the hollow guidewire or conventional guidewire has reached the target site. The drive shaft as disposed within the axial lumen of the hollow guidewire and extending distally from the guidewire distal end may be rotated, preferably oscillating between a set number of rotations into the occlusion. In an embodiment, the distal tip of the core element may be configured for further advancement and/or retraction from the distal end of the hollow guidewire, such that the position of the hollow guidewire and catheter system can be maintained and stabilized while the drive shaft is rotated and translated out of the axial lumen of the hollow guidewire.

The distal tip of the core element may be coiled, blunted, flattened, enlarged, twisted, basket shaped, football shaped, bullet shaped, or the like. In some embodiments, to increase the rate of removal of the occlusive material, the distal tip is sharpened or impregnated with an abrasive material such as diamond chips, diamond powder, glass, or the like. The core element distal tip may be formed of any suitable material such as stainless steel, nitinol, cobalt-chromium, polymeric material, or radiopaque material such as platinum-iridium. In an embodiment, the core element distal tip may be formed from a composite material such as a stainless steel tip having a cavity filled with a radiopaque material. Alternatively, or in addition thereto, the plaque removal assembly may comprise a laser, an RF electrode, a heating element (e.g., resistive element), an ultrasound transducer, or the like. A lead of the plaque removal assembly may extend proximally through the axial lumen of the hollow guidewire body. In an embodiment, the drive shaft is distally tapered, as for example along the deflected distal end of the guidewire body.

The hollow guidewire body includes proximal and distal portions. In an embodiment, the elongate hollow guidewire body may be formed from a unitary tube having different portions. Alternatively, the guidewire body may be formed from several members joined longitudinally to one another forming the various portions. In an embodiment, the distal portion of the guidewire body comprises one or more patterns such as, but not limited to, interrupted helical pattern and ribbed pattern. Either of the patterned portions may extend proximally from the distal end of the hollow guidewire body with the other pattern extending proximally from a proximal end of the other. Alternatively, the guidewire distal portion may comprise a single type of pattern. In an embodiment, the interrupted helical patterned portion comprises laser edged helical windings formed at 180° interrupted by 30° segments. In an embodiment, the one or more patterned portions, together, have a longitudinal dimension ranging from about 0.3 to about 10 cm, from about 1 to about 5 cm, normally about 4 cm. In an embodiment, all or at least a portion of the deflected distal portion may be plated with suitable radiopaque material, such as gold.

In an embodiment, the guidewire body comprises a hollow solid walled tube. A proximal coil section may be longitudinally disposed between a distal end of the solid walled tube and the patterned distal portion of the guidewire body. The patterned distal portion may be formed, as discussed above, from one or more patterns such as an interrupted helical pattern and ribbed pattern portions. In an embodiment, the proximal coil and the patterned distal portion, together, form a flexible distal section having a longitudinal dimension ranging from about 1 to about 200 cm, from about 20 to about 50 cm, normally about 30 cm. The one or more patterned portions at the guidewire distal portion and the proximal coil may be independently formed from suitable material such as stainless steel, nitinol, polymeric material, or radiopaque material such as platinum-iridium or cobalt-chromium.

In an embodiment, an elongate tube extends within at least a portion of the guidewire axial lumen. In an embodiment, the elongate tube is coupled to the guidewire body distal end. The elongate tube may be distally tapered at the distal end. The elongate tube tapered distal end may be in the form of a ribbon. The tapered distal end may have a flat or arcuate (e.g., crescent shape) transverse profile. In an embodiment, the elongate tube is skived at the distal end to provide the tapered distal end. The elongate tube generally has a longitudinal dimension ranging from about 1 to about 200 cm, from about 20 to about 190 cm, normally about 170 cm.

In an embodiment, the elongate tube is tapered along the length of the flexible distal section of the hollow guidewire. In an embodiment, the tapered elongate tube terminates proximally at the proximal end of the flexible distal section. In an embodiment, the proximal end of the tapered elongate tube terminates within a solid tube which extends to the hollow guidewire proximal end. A distal end of the solid tube may form a distal flange extending over the proximal end of the elongate tube forming a joint (e.g., a lap joint) therewith.

The one or more portions of the elongate tube may be independently formed from any suitable material such as stainless steel, nickel-titanium alloy (such as nitinol), radiopaque material (such as platinum-iridium material), cobalt chromium, polymer (such as PEEK), or any combination thereof.

In an embodiment, an inner coil is disposed about the distal portion of the drive shaft radially separating it from the elongate tube. In an embodiment, the inner coil extends along the tapered distal portion of the elongate tube. The inner coil may be formed from any suitable material such as stainless steel, nickel-titanium alloy (such as nitinol), radiopaque material (such as platinum-iridium material), cobalt chromium, or any combination thereof. The inner coil may have a longitudinal dimension ranging from about 1 to about 50 millimeter (“mm”), from about 2 to about 10 mm, normally about 4 mm. In an embodiment, the inner coil extends distally about the drive shaft to the tapered distal end of the elongate tube.

The drive shaft may be of a single wire type, a counter-wound guidewire construction, or be formed from a composite structure comprising a fine wire around which a coil is wrapped. In an embodiment, at least a portion of the drive shaft may be coated with lubricious material to enhance its movement within the inner lumen of the body.

The dimensions of the hollow guidewires of the present invention may vary depending on the target lumen, with the body and the specific needs of the procedure. In an embodiment, the radial dimension (e.g., outer diameter) of the guidewire body ranges from about 0.040 to about 0.008 inches (“in.”), from about 0.035 to about 0.008 in., from about 0.024 to about 0.008 in., normally from about 0.018 to about 0.009 in. A wall thickness of the hollow guidewires of the present invention typically range from about 0.001 to about 0.004 in., but as with the other dimensions may vary depending on the desired characteristics of the hollow guidewire.

Systems and kits of the present invention may include a support system or access system, such as a catheter, having a body adapted for intraluminal introduction to the target blood vessel. The dimensions and other physical characteristics of the access system body will vary significantly depending on the body lumen which is to be accessed. The body of the support or access system is very flexible and is suitable for introduction over a conventional guidewire, or the hollow guidewire (e.g., having a removable handle) of the present invention. The support or access system body can either be for “over-the-wire” introduction or for “rapid exchange,” where the guidewire lumen extends only through a distal portion of the access system body. Optionally, the support or access system can have at least one axial channel extending through the lumen to facilitate infusion to and/or aspiration of material from the target site. Support or access system bodies will typically be formed from an organic polymer, such as polyvinylchloride, polyurethanes, polyesters, polytetrafluoroethylenes (PTFE), silicone rubbers, natural rubbers, or the like. Suitable bodies may be formed by extrusion, with one or more lumens that extend axially through the body. For example, the support or access system can be a support catheter, interventional catheter, balloon dilation catheter, atherectomy catheter, rotational catheter, extractional catheter, laser ablation catheter, guiding catheter, stenting catheter, ultrasound catheter, and the like. The support system, which is described in more detail in commonly owned U.S. patent application Ser. No. 10/864,075, filed Jun. 8, 2004, the disclosure of which is incorporated herein by reference in its entirety, may be used for over-the-wire introduction or for rapid exchange.

The position of the hollow guidewire and/or support system may be maintained and stabilized during the advancing of the distal tip of the drive shaft. At the end of the plaque removal, the method may further comprise exchanging the hollow guidewire with the conventional guidewire. Additionally, other features of the devices of the present invention and methods using the same, are further described in commonly owned U.S. patent application Ser. No. 11/236,703, filed Sep. 26, 2005, and assigned to the assignee of the present invention, the disclosure of which is incorporated herein by reference in its entirety. In an embodiment, when the handle assembly is removably attached to the hollow guidewire, the handle assembly may be detached from the hollow guidewire (e.g., with the use of a guidewire extension) and the support catheter is removed and exchanged with another support catheter.

In an embodiment, the proximal end of the elongate member is housed within a handle assembly with proximal and distal ends, and a housing disposed therebetween. At the distal end, the handle assembly includes a strain relief having a lumen extending therethrough. A torquer with a lumen is disposed between the strain relief and the housing. The proximal end of the guidewire with the drive shaft proximal end disposed through the guidewire lumen, extends through the strain relief and the torquer. The proximal end of the guidewire terminates and is secured in place within a connector assembly which is located within the housing. The connector assembly limits the motion of the elongate member while allowing the drive shaft to either or both rotationally oscillate and translate within the elongate member. The proximal end of the drive shaft extends proximally from the connector assembly and is secured by a shaft coupling within the housing. In an embodiment, a motor disposed within the housing provides rotational oscillation to the drive shaft during operation. A connector cable connects the motor for moving (i.e., oscillate, rotate, translate, reciprocate, vibrate, or the like) the drive shaft and its distal tip, to a control system and power supply. It should be appreciated that the various components may be located within or outside of the housing. By way of example, the control system may be placed within the housing. Similarly, the power supply may be battery operated and similarly and entirely locatable within the housing.

The handle assembly may be removably or fixedly attached to the proximal ends of the hollow guidewire and the drive shaft. Optionally, some embodiments of the connector assembly include an aspiration or infusion port (not shown) for facilitating fluid exchange (e.g., delivery or removal) at the target site through the axial lumen.

Torque transmission of the guidewire body and activation of the core element may be carried out sequentially or simultaneously as a physician steers through a tortuous blood vessel. This can advantageously be accomplished while maintaining the handle in a stationary configuration that is ergonomically easy to grasp and control. The handle may further comprise a drive motor to move (e.g., oscillate, reciprocate, translate, rotate, vibrate, or the like) the core element, actuators for steering the guidewire body, a control system including circuitry which provides feedback control as discussed in more detail below, and/or a power supply. The handle may alternatively be removably coupled to the guidewire body as described above. An optional polymeric insert may be provided as part of a coupling to reduce electrical emission during operation of the device.

The plaque removal assembly may be fixedly or movably disposed at the distal end of the hollow guidewire body. If the plaque removal assembly is movable, the plaque removal assembly may be movable from a first axially retracted position (or extending distal to the hollow guidewire body) to a second position which is longitudinally distal to the first position. The drive shaft of the present invention may be axially movable and rotatable within the axial lumen of the hollow guidewire body. In an embodiment, either or both the guidewire and the drive shaft may be coated with any one or more or combinations of hydrophilic coatings and therapeutic agents. In an embodiment, the guidewire is coated with heparin or other similar therapeutic agents. In an embodiment, the drive shaft may be coated with Teflon® or other materials to improve the rotation of the drive shaft within the guidewire axial lumen.

In use, the access system can be delivered to the target site over a conventional guidewire. Once the access system has been positioned near the target site, the conventional guidewire can be removed and the elongate member (e.g., hollow guidewire) of the present invention can be advanced through an inner lumen of the access system to the target site. Optionally, the support system can be delivered concurrently with the advancement of the hollow guidewire. Alternatively, because the elongate member can have the flexibility, pushability, and torqueability to be advanced through the tortuous regions of the vasculature, the elongate member may be advanced through the vasculature to the target site without the use of the separate guidewire. In such embodiments, the access system can be advanced over the elongate member of the present invention to the target site. Once the elongate member has been positioned at the target site, the drive shaft is rotated, preferably, in an oscillation rotational mode, and advanced into the occlusive material or the entire elongate member may be advanced distally into the occlusion. The rotation of the drive shaft distal tip creates a path forward of the elongate member. In some embodiments, the path created by the distal tip has a path radius which is larger than the radius of the distal end of the elongate member. In other embodiments, the path created by the distal tip has a path radius which is the same size or smaller than the radius of the elongate member.

The hollow guidewire device can be used in conjunction with conventional guidewires to cross a total occlusion. For example, the hollow guidewire can be used to cross calcified regions (e.g. proximal and distal cap) of the total occlusion requiring more penetration force. A conventional guidewire can be used to cross softer, more tortuous regions of the occlusion that require more flexibility. The hollow guidewire and conventional guidewire can be placed parallel as they are advanced or can be exchanged through one access system. If one guidewire enters sub-intimal space, it may be left in place while another hollow guidewire or conventional guidewire continues advancement in parallel.

The preferred operating mode of rotational oscillation of the drive shaft and the distal tip is of particular benefit to the present invention as it prevents tissue from wrapping around the distal tip of the plaque removal drive shaft. This in turn allows for enhanced penetration through, in and/or out of the occlusive or stenotic material. In an embodiment, the drive shaft is configured for rotational oscillation movement such that the shaft distal tip may be rotated through an angle equal to or less than 360°. The shaft distal tip is then adapted to rotate back in the same manner and amount. In an embodiment, the during each oscillation cycle, the motor is configured to provide from about 100 to about 200,000 revolutions per minute (“rpm”); from about 5,000 to about 50,000 rpm; normally about 12,000 rpm. Typically, the drive shaft is oscillated so that it changes polarity after a period of time. The period of time may range from about 0.2 to about 5.0 seconds, usually in a range from about 0.3 to about 1.2 seconds, and normally about 0.7 seconds. By way of example, in an embodiment, the motor is configured to provide about 140 complete cycles (i.e., rotations of 360°) per about every 0.7 seconds before it oscillates to change the polarity of the rotation.

Advancing may further comprise reciprocating axial translation of the distal tip of the drive shaft so as to completely cross the total occlusion. Oscillation and reciprocation of the drive shaft may be carried out sequentially or simultaneously. Generally, oscillation and/or reciprocation movement of the drive shaft are carried out by a drive motor. However, a device operator may also easily affect reciprocation by simply axially translating the device by its handle manually. Advancing may further comprise extending the drive shaft from a retracted configuration to an extended configuration relative to the distal portion of the hollow guidewire body, wherein the drive shaft is simultaneously or sequentially extended and oscillated.

Proper positioning at the occlusion site may further be verified by viewing a distal end of the hollow guidewire under fluoroscopy via any of the radiopaque components of the devices, such as the inner coil, or the core element distal tip.

Electronic circuitry within the control system of the handle may measure a variety of characteristics for feedback control. For instance, the load encountered during advancement of the distal tip in the body lumen may be measured. For example, a load sensor may be coupled to the motor and configured to provide an output representative of the load on the motor. In an embodiment, an audible and/or visual output may be coupled to the load sensor to provide load status to the user. The audio feedback may be represented in a continuous spectrum or it may be represented as a plurality of discrete load levels. The visual feedback may be represented as a plurality of discrete load levels. In another embodiment, absence of load may be indicative of a break or fracture in the oscillating drive shaft distal tip. A locking mechanism on a distal end of the guidewire body may be provided to further prevent inadvertent release of the distal tip of the drive shaft into the body lumen by locking it to a distal end of the hollow guidewire. Still further, the device may be automatically disabled in response to the no load measurement as an added safety feature. In still another instance, a use of the device based on time or number of revolutions or oscillations may be measured. The device may be automatically and permanently disabled once the measured time or number is above a threshold value. This safety feature protects against device fatigue and warrants that the device is not operable past its optimal lifetime use.

In an embodiment, the present invention provides a kit. The kit has any of the hollow guidewires and/or the drive shafts described herein and instructions for use according to any of the methods described herein. The instructions for use in passing occlusions or stenosis in a body lumen comprise rotational oscillation and advancing either or both the hollow guidewire and the drive shaft into the occlusive or stenotic material to create a path through the occlusive or stenotic material. A package is adapted to contain either or both the hollow guidewire, the core element, and the instructions for use. In some embodiments, the instructions can be printed directly on the package, while in other embodiments the instructions can be separate from the package.

These and other features of the invention will be further evident from the attached drawings and description of the invention.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 is an elevational view of a system embodying features of the present invention having a guidewire with a pre-determined deflected distal tip.

FIG. 2 is an elevational view of an exemplary guidewire embodying features of the present invention and having a metal wire at a distal end.

FIG. 3 is an elevational view of an exemplary guidewire embodying features of the present invention and having an elongate tube.

FIG. 4 is an elevational view of an exemplary guidewire embodying features of the present invention having multiple portions including a proximal coil portion.

FIG. 5 is an elevational view of an exemplary guidewire embodying features of the present invention having multiple portions.

FIG. 6A is an elevational view of an exemplary guidewire embodying features of the present invention and having an elongate tube coupled to a solid proximal tube.

FIG. 6B is an elevational view of an exemplary guidewire embodying features of the present invention having a metal wire at the distal end and an elongate tube coupled to a solid proximal tube and a distal elongate body.

FIG. 7 is an enlarged view of a distal end of a drive shaft with a composite distal tip.

FIG. 8 is a handle/torque assembly embodying features of the present invention.

FIG. 9 is an elevational view of an exemplary guidewire embodying features of the present invention with the drive shaft distally extending beyond the shaft distal tip.

FIG. 10 is an elevational view of an exemplary guidewire embodying features of the present invention and having an elongate tube attached to a solid tube.

### DETAILED DESCRIPTION OF THE INVENTION

The systems, devices and methods according to the present invention will generally be adapted for the intraluminal treatment of a target site within a body lumen of a patient, usually in a coronary artery or peripheral blood vessel which is occluded or stenosed with atherosclerotic, stenotic, thrombotic, or other occlusive material. The systems, devices and methods, however, are also suitable for treating stenoses of the body lumens and other hyperplastic and neoplastic conditions in other body lumens, such as the ureter, the biliary duct, respiratory passages, the pancreatic duct, the lymphatic duct, and the like. Neoplastic cell growth will often occur as a result of a tumor surrounding and intruding into a body lumen. Removal of such material can thus be beneficial to maintain patency of the body lumen. While the remaining discussion is directed at passing through atheromatous or thrombotic occlusive material in a coronary artery, it will be appreciated that the systems and methods of the present invention can be used to remove and/or pass through a variety of occlusive, stenotic, or hyperplastic material in a variety of body lumens. It should be appreciated, that many of the features of the different embodiments as described, may be used in the described embodiment, alone, or together with others.

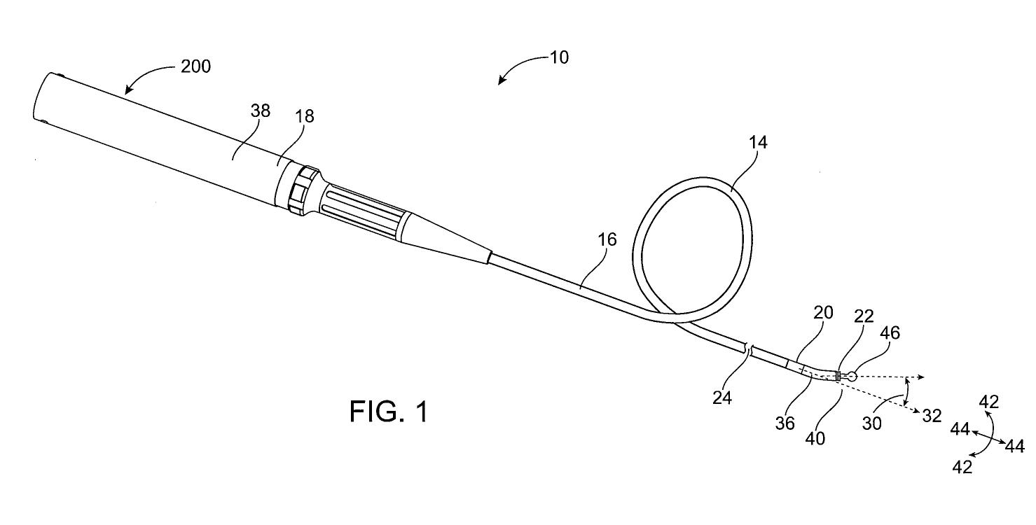

An apparatus 10 embodying features of the present invention is illustrated in FIG. 1 generally including an elongate member 14, such as a guidewire, with a proximal portion 16, a proximal end 18, a distal portion 20, a distal end 22, and an axial lumen 24 extending therethrough. A handle assembly 200 may be fixedly or removably attachable to the elongate member 14. In an embodiment as shown, the handle is fixedly attached to the elongate member.

The distal portion 20 of the elongate member 14, has a pre-determined fixed deflection 30, as compared to a longitudinal axis 32 of the elongate member 14 (i.e., a deflection angle as defined by the tangential line formed between the guidewire distal end 22 of the elongate member 14 and the longitudinal axis 32). The distal end deflection is designed such that when the guidewire 14 is housed within and introduced through another elongate body, such as a balloon catheter, the angle of the deflected distal tip may at least be partially decreased (e.g., straightened) to accommodate the inner diameter of the catheter. Once the guidewire (or its distal end) exits the catheter (e.g., balloon catheter), the guidewire distal tip returns to its preset deflection angle. The pre-determined fixed deflection 30, generally, ranges from about 0 to about 90 degrees (“°”), usually from about 0 to about 60°, and normally from about 5 to about 45°. In an embodiment, the pre-determined fixed deflection is about 15°, about 30°, or about 45°. The deflection 30 of the distal end 22 of the elongate member 14 may be arrived at in a smooth transition or in an abrupt transition, or any type and degree of transition inbetween.

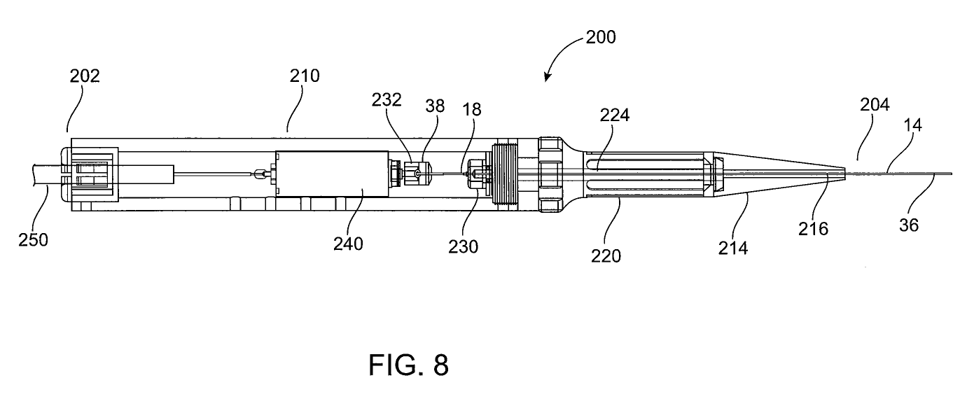

The apparatus 10 may further comprise a plaque removal assembly, such as a rotatable drive shaft 36, for removing tissue and creating a path through the body lumen. The drive shaft 36 has a shaft proximal end 38 (as best can be seen in FIG. 8) and a shaft distal end 40 and is received within the axial lumen 24 of the hollow guidewire 14. In an embodiment, the drive shaft is configured for either or both rotational (with or without oscillation) and axial movement, as for example shown by arrows 42 and 44. In an embodiment, the drive shaft may be configured for rotation (with or without oscillation) but not axial movement. A distal tip 46 of the drive shaft 36 at the shaft distal end 40 may have a shaped profile, enabling the movement or positioning of the distal tip 46 beyond the distal end 22 of the hollow guidewire 14. The rotation of the drive shaft 36 may be used to create a cutting path forward of the distal end 22 of the hollow guidewire for passing through the occlusive or stenotic material in the body lumen. The drive shaft 36 and the distal tip 46, may independently be formed from stainless steel or nitinol, or other suitable material including other radiopaque materials such as platinum/tungsten compounds. The proximal end 18 of the hollow guidewire 14 may be coupled to a vacuum source or a fluid source (not shown) such that the target site can be aspirated or infused during the procedure, if desired.

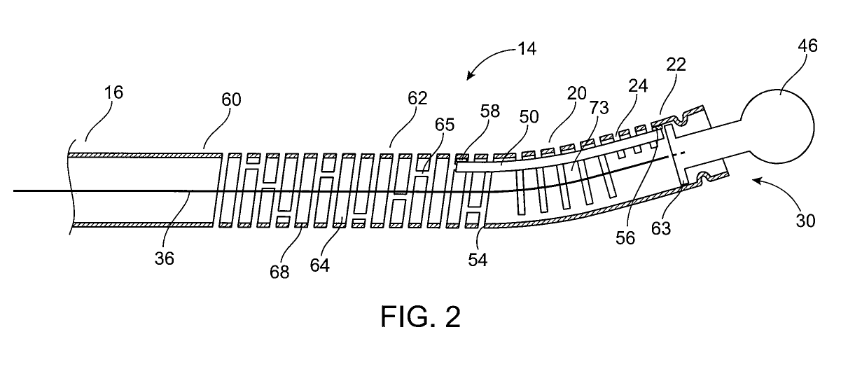

In an embodiment, features of which are shown in FIG. 2, the pre-determined fixed deflection 30 is, at least in part, achieved by way of an elongate body such as a metal wire or ribbon 50 longitudinally disposed within the distal portion 20 of the hollow guidewire inner lumen 24 and is fixedly attached to an inner surface 54 thereof. The elongate body may have a flat or arcuate (e.g., crescent shape) transverse profile. In the embodiment shown, the metal wire or ribbon 50 is attached to the inner lumen 24 along at least a distal attachment point 56 at the hollow guidewire distal end 22 and at a proximal attachment point 58 proximally extending from the guidewire distal end 22. The elongate body 50 (such as metal wire or ribbon) may be formed from suitable material such as stainless steel, nickel-titanium, or cobalt-chromium; and has a longitudinal dimension ranging from about 0.3 to about 6 centimeters (“cm”), from about 0.5 to about 2 cm. In an embodiment, the metal wire or ribbon 50 has a longitudinal dimension of about 1 cm. In an embodiment, the pre-determined fixed deflection is, at least in part, achieved by way of a shaped distal portion of the guidewire body. For example, the shaped distal portion may be made from a nickel-titanium alloy and heat set to the pre-determined fixed deflection angle. In such an embodiment, the distal portion may, optionally, also include the metal wire or ribbon as further means to provide the pre-determined fixed deflection. In an embodiment, the drive shaft is distally tapered, as for example along the deflected distal end of the guidewire body.

In the embodiment shown, the hollow guidewire 14 is formed from a unitary construction formed from a single hypotube 60 including the proximal portion 16, the distal portion 20, and an intermediate portion 62 disposed therebetween. The drive shaft distal tip may include a lock feature 63 to minimize the unwanted detachment of the drive shaft distal tip from the guidewire distal end 22 (e.g., in the event of drive shaft fracture). At least a portion of the hypotube 60 may be laser edged to create a plurality of helical windings or spirals 64. The laser cuts may extend all the way from the hollow guidewire proximal end to the distal end or the laser cuts may extend through less than all of the length of the hypotube, usually the distal portion 20 and the intermediate portion 62. The laser cuts used to create the helical windings 64 may extend completely through a wall 68 of the hypotube or may extend only partially through the hypotube wall so as to create thinner wall portions (e.g., grooves). In the embodiment shown due, at least in part, to the integral formation of the distal portion 20, the intermediate portion 62, and the proximal portion 16, there are no joints. A radiopaque marker may be disposed at the distal portion 20 of the hollow guidewire 14, usually at the distal end 22, to enhance visualization of the distal end during the procedure.

The laser edging removes at least a portion of the material from the guidewire body 14. The laser cuts 64 may be, as shown, in the form of an interrupted helical pattern ranging from about 90° to about 270°, preferably about 180°. Interruptions or breaks 65 have no laser cuts and are in a range from about 5° to about 225°, preferably 30° segments. Significantly, the interruptions 65 help preserve the integrity and continuity of the device 10, particularly when it is steered through tortuous blood vessels. The interrupted helical pattern may have a clockwise or counterclockwise helical direction and a kerf ranging from about 0.0005 inches (“in.”) to about 0.0040 in. The helical windings 64 may have the same or variable pitch through at least one section of the intermediate and distal portions, 62 and 20. As can be appreciated, the pitch between adjacent windings will affect the flexibility of hypotube 60 and the pitch may be selected to effectuate the desired characteristics of the hollow guidewire 14. As can be appreciated, the hollow guidewire 14 may comprise any number of sections, and the sections in turn may have any desired pitch or kerf, any number or degree of helical windings or interruptions, clockwise or counterclockwise helical directions, any length, or variations thereof.

As further shown, the distal portion 20 of the guidewire may comprise a different patterned section and radial slots, openings, and/or thinned portions 73. The slots 73 may extend along about a distal length of the guidewire body ranging from about 1 millimeter (“mm”) to about 20 mm, normally about a 4 mm distal length of the guidewire body 14. It will be appreciated that this section may be shorter or longer, as desired. The radial slots/openings 73 may be formed on the guidewire body 14 by way of laser edging or electro-discharge machining (edm) that removes at least a portion of the material from the guidewire body, as described above with respect to the helical windings. The slots/openings 73 may extend around less than the entire circumference of the hypotube, typically extending between about 25% (e.g., 90°) to about 90% (e.g., 324°) of the guidewire body. Support ribs typically will extend between 100% (e.g., 360°) to about 25% (e.g., 90°) around the circumference of the hollow guidewire body 14.

The pitch between helical windings 64 may decrease in the distal direction so as to provide the hollow guidewire 14 with increasing flexibility in the distal direction. In an embodiment, it may be desirable to have sections of the guidewire to have no helical cuts or have laser cuts that have a pitch that increases in the distal direction so as to provide less flexibility over a portion of the hollow guidewire. The less flexible portion may be at the proximal portion, the intermediate portion, or at the distal portion including at or near the distal end of the hollow guidewire, or any combination thereof. As described above, in reference to FIG. 1, the drive shaft 36 is disposed within the axial lumen 24 of the guidewire body 14 with the shaft distal tip 46 extending distally from the distal end 22 of the guidewire body 14.

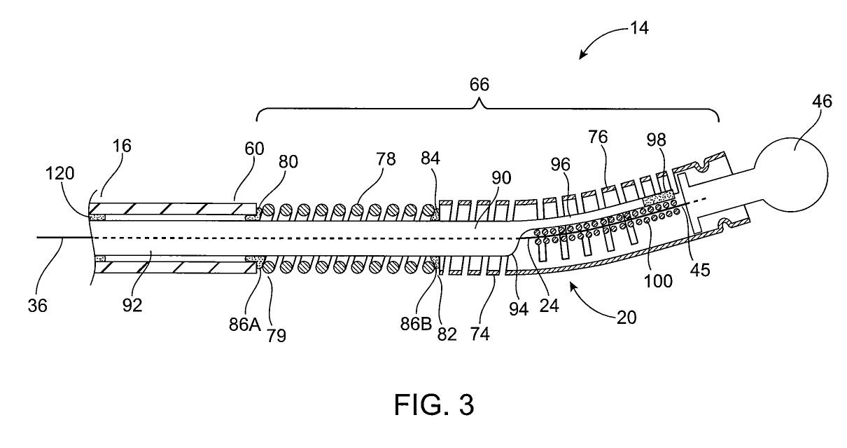

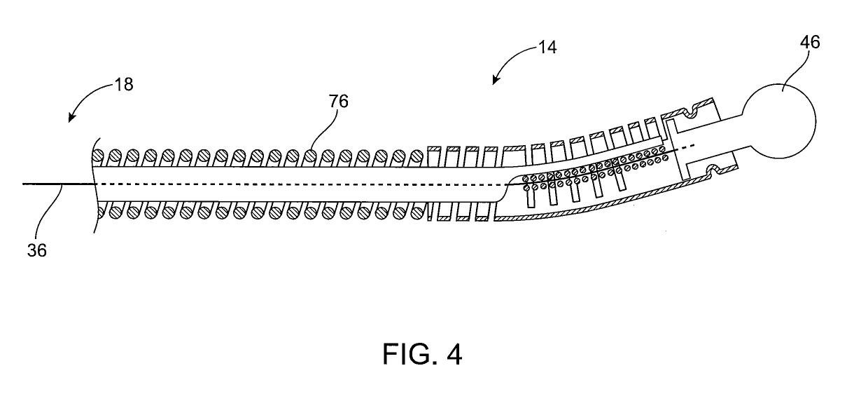

In an embodiment, features of which are shown in FIG. 3, the hollow guidewire 14 includes the proximal portion 16 including a proximal tube 60 and a flexible distal portion 66 including an intermediate coil 74 and a distal coil 76 with a proximal coil 78 disposed between the distal end of the tube 60 and the proximal end of the intermediate coil 74. In some embodiments, the proximal tube 60, the proximal coil 78, the intermediate coil 74, and the distal coil 76 are, independently, formed from stainless steel, nitinol, polymeric material, radiopaque material including platinum such as platinum/iridium compounds, or a combination thereof. In an embodiment, the flexible distal portion 66 may have a longitudinal dimension ranging from about 1 to about 200 cm, from about 10 to about 80 cm, from about 20 to about 40 cm, normally about 35 or about 30 cm. In an embodiment, the deflected distal portion 20 of the guidewire member 14 extends from about 0.3 to about 10 cm, usually from about 1 to about 5, normally about 4 cm. In an embodiment, all or at least a portion of the deflected distal portion 20 may be plated with suitable radiopaque material, such as gold. Alternatively, as shown in FIG. 4, the proximal coil 78 may extend proximally to the proximal end 18 of the hollow guidewire 14.

Now, referring back to FIG. 3, the proximal coil 78; at a proximal end 79, is engaged with a distal end 80 of the proximal tube 60; and at a distal end 82 with a proximal end 84 of the intermediate coil 74. The engagement of the proximal coil 78 with the intermediate coil 74 and the proximal tube 60 may be by way of one or more independently selected ways, such as threading, soldering, and adhesive. As shown, the proximal coil is engaged by way of solders 86A and 86B at its two proximal and distal ends.

As shown, an elongate tube 90 is disposed along at least a portion of the axial lumen 24 of the hollow guidewire 14. The elongate tube 90 has a proximal portion 92 and a relatively short distal portion 94. The distal portion 94 of the elongate tube 90 may include a shaped distal end, such as a tapered distal end, generally, in the form of a ribbon 96 extending distally to a proximal end 45 of the drive shaft distal tip 46. The ribbon 96 may have a flat or arcuate (e.g., crescent shape) transverse profile. In an embodiment, the elongate tube is skived to provide the tapered distal end. The elongate tube 90 may be formed from any suitable material, such as nitinol hypotube. The distal end of the elongate tube 90 is attached to the distal portion 20 of the hollow guidewire 14 by suitable means, such as solder 98. The elongate tube 90 at a proximal end may be fixedly joined to the tube 60 by suitable means such as solder 120. The elongate tube is further attached to the distal end 80 of tube 60 and the proximal end 84 of the intermediate coil 74, by suitable means such as solders 86A and 86B, respectively. In an embodiment, the attachment of the elongate tube 90 to the proximal end of the intermediate coil 74 and at the distal end of the distal coil 76, by suitable means such as solders 86B and 98, enables the setting of the deflection as is shown in FIG. 3. The elongate tube 90 generally has a longitudinal dimension ranging from about 1 to about 200 cm, from about 20 to about 180, normally from about 30 to about 170 cm. The untapered portion of the elongate tube 90 has an outer diameter ranging from about 0.005 to about 0.040 inches (“in.”), from about 0.008 to about 0.018 in., normally about 0.009 in.

Optionally, and as shown, an inner coil 100 is disposed around, and extends proximally from, the distal end of the drive shaft 36. The inner coil 100 radially separates the distal portion of the drive shaft from the distal end of the elongate tube 90. The inner coil 100 is preferably formed from a radiopaque material so as to provide a radiopaque marker for fluoroscopic tracking of the hollow guidewire 14. The radiopaque coil 100 may be formed from suitable material including platinum compounds such as platinum-iridium coil. The radiopaque inner coil 100 may be soldered, glued, or otherwise attached to the elongate tube 90. In an embodiment, the inner coil 100 may float without being fixedly attached to the elongate tube. The inner coil 100 may have any desired length and pitch. In an embodiment, the inner coil 100 has a longitudinal dimension substantially the same as that of the deflected distal portion 20 of the hollow guidewire 14.

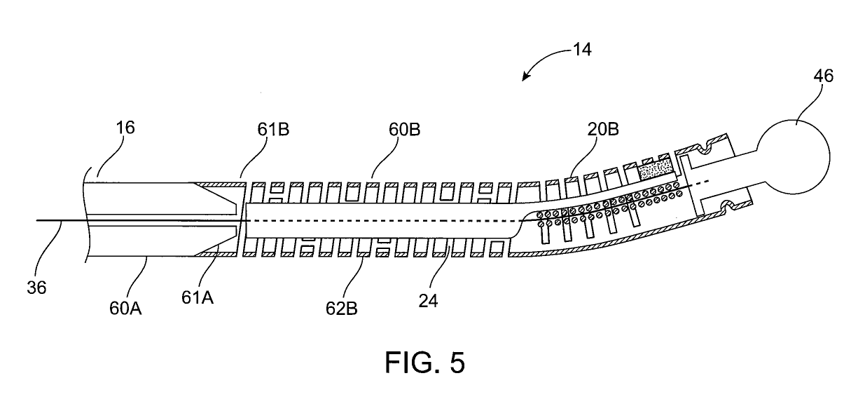

In an alternate embodiment, features of which are shown in FIG. 5, the hypotube 60 may comprise at least two portions, a proximal solid section 60A and a relatively short distal section 60B including intermediate portion 62B and distal portion 20B. A distal end 61A of the proximal section 60A may, as shown, be distally tapered and fixed within the inner surface of the guidewire lumen 24 to a proximal end 61B of the distal section 60B, by way of suitable means such as welding or soldering.

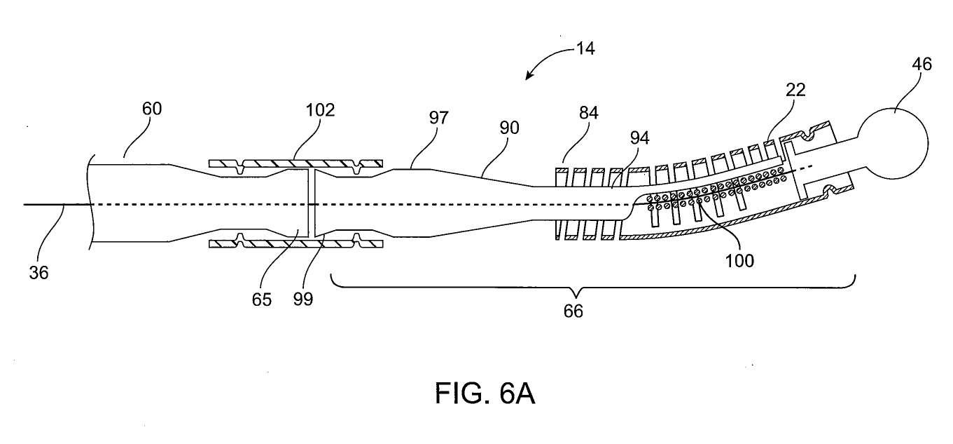

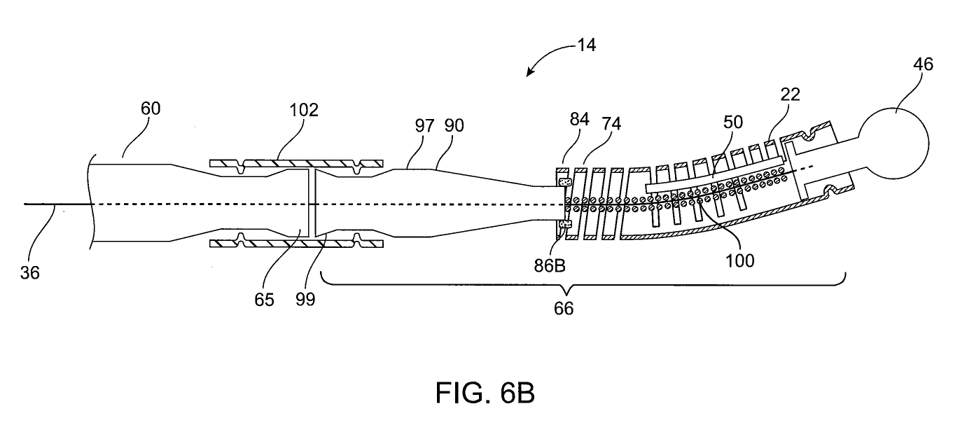

In an embodiment, features of which are shown in FIG. 6A, the an intermediate portion 97 of the elongate tube 90 which extends proximal the elongate tube shaped distal end 94, may be further distally tapered. In an embodiment, the tapered intermediate portion 97 extends along substantially the length of the flexible distal portion 66 and has a longitudinal dimension ranging from about 20 to about 60 cm, usually about 35 or about 30 cm. The elongate tube 90, when tapered, as for example in the intermediate portion 97, has an outer diameter ranging from about 0.005 to about 0.040 in., from about 0.008 to about 0.018 in., normally about 0.011 in. In the embodiment, features of which are shown in FIG. 6A, the elongate tube 90 at its proximal end 99 is joined to the distal end 65 of the solid wall tube 60. As shown, a cuff 102, surrounds the two ends, of the elongate tube and solid wall tube, press fitting or soldering the elongate tube and the proximal solid wall tube to one another. The cuff 102 may be formed from suitable material such as stainless steel, nickel-titanium, or platinum-iridium. Additionally, the elongate tube 90 may be at least partially covered with a coil or polymer (such as PEBAX). In an alternate embodiment, as shown in FIG. 6B, the elongate tube 90 terminates at the proximal end 84 of the intermediate coil 74 and is fixedly attached thereto by suitable means such as the solder 86B. The inner coil 100, as shown, extends proximally beyond the proximal end of the elongate body 50 to the proximal end 84 of the intermediate coil 74.

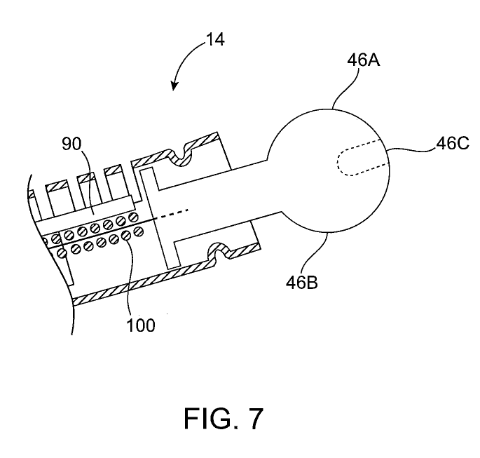

As described above with reference to FIG. 1, the drive shaft 36 is disposed within the axial lumen 24 of the guidewire body 14 with the shaft distal tip 46 extending distally from the distal end 22 of the guidewire body 14. The distal tip 46, in an embodiment as shown in FIG. 7, may be a filled-tip 46A, with the tip body 46B formed from stainless steel or nickel-titanium and a tip end 46C formed from a radiopaque material, such as a platinum-tungsten compound. The radiopaque material of the tip end 46C may be disposed within the tip body 46B by suitable means such as solder or swaging.

Now referring back to FIG. 1 and as best seen in FIG. 8, the proximal end 18 of elongate member 14 is housed within handle assembly 200. The handle assembly 200 has proximal and distal ends, 202 and 204, and a housing 210 disposed therebetween. At the distal end 204, the handle assembly 200 includes a strain relief 214 having a lumen 216 extending therethrough. A torquer 220 with a lumen 224 is disposed between the strain relief 214 and the housing 210. The proximal end 18 of the guidewire 14 with the drive shaft proximal end 38 disposed through the guidewire lumen 24, extends through the lumen 216 of the strain relief 214 and lumen 224 of the torquer 220. The proximal end 18 of the guidewire 14 terminates and is secured in place within a connector assembly 230 which is located within housing 210. The connector assembly 230 limits the motion of the elongate member 14 while allowing the drive shaft 36 to rotate and translate within the elongate member 14. The proximal end 38 of the drive shaft 36 extends proximally from the connector assembly 230 and is secured in the housing 210 by shaft coupling 232. A motor 240 disposed within the housing 210 provides rotational oscillation to the drive shaft during operation. A connector cable 250 connects the motor 240, for moving (i.e., rotate, oscillating, translate, reciprocate, vibrate, or the like) the drive shaft and the shaped distal tip 46 of the drive shaft 36, to a control system (not shown) and power supply (not shown). It should be appreciated that the various components may be located within or outside of housing 210. By way of example, the control system may be placed within the housing 210. Similarly, the power supply may be battery operated, and similarly and entirely locatable within housing 210.

Optionally, some embodiments of the connector assembly 230 includes an aspiration or infusion port (not shown) for facilitating fluid exchange (e.g., delivery or removal) at the target site through the axial lumen 24. A polymer insert, may further be disposed within shaft coupling 232, used as part of a coupling of the drive shaft to the motor 240 to reduce electrical emissions during operation.

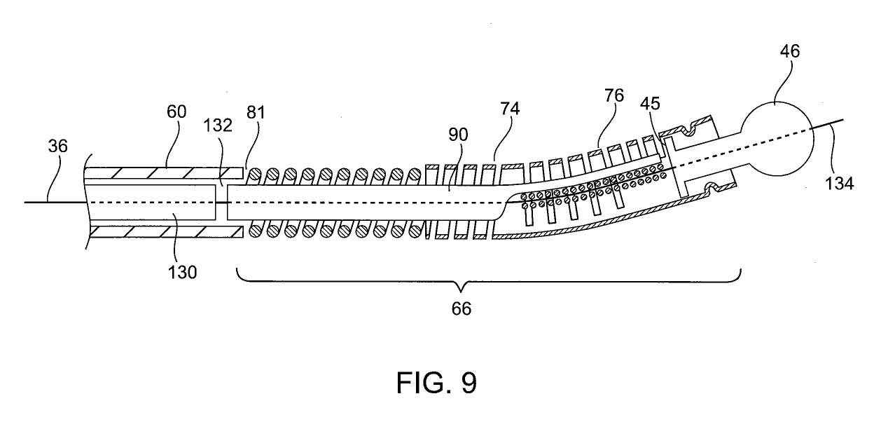

Now turning to FIG. 9, wherein like references refer to like elements, the elongate tube 90 extends from the proximal end 45 of the drive shaft distal tip 46 to a proximal end 81 of the flexible portion 66. An optional tubular member 130, as shown, may be disposed proximal the elongate tube 90 within the tube 60. The distal end of the tubular member 130 and the proximal end of the elongate tube 90 may be longitudinally separated by a gap 132, or form a joint such as a butt-joint or a lap-joint. The optional tube 130 may be formed of suitable material such as stainless steel, nitinol, or polymeric material including PEEK (polyetherketone).

In an embodiment, as shown, the distal end of the drive shaft 36 may have a distal extension 134 extending distally from the distal end of the distal tip 46, thereby, helping the navigation of the drive shaft within the target lumen. To enhance the radiopacity of the guidewire member 14 at its distal end, the intermediate portion 74, the distal portion 76, and the drive shaft distal tip 46, may be formed from or plated with radiopaque material such as cobalt-chromium or gold. In an embodiment, the inner coil 100 may be formed from a polymeric material or eliminated in total. In an embodiment, the drive shaft 36 may be coated with coating suitable for its use such as hydrophilic, or hydrophobic coatings.

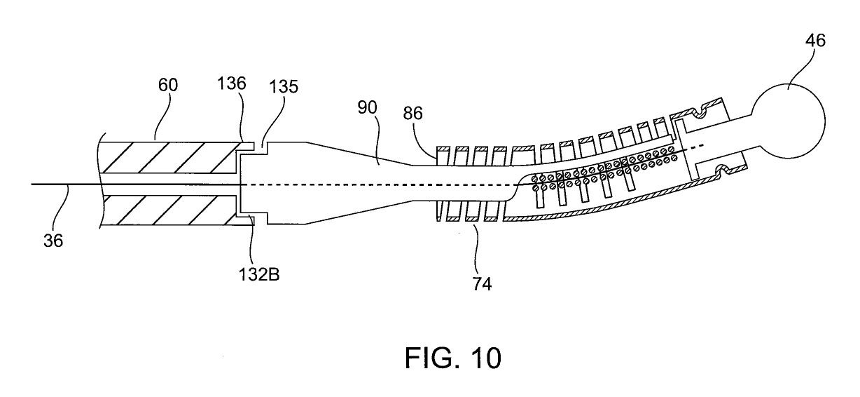

In an embodiment, features of which are shown in FIG. 10, the elongate tube 90, extends proximally from the proximal end 45 (shown in FIG. 3) of the drive shaft distal tip 46 beyond the proximal end 86 of the intermediate portion 74. The elongate tube 90 tapers at a proximal end forming an undercut 135 and is fixedly disposed within the distal end of the proximal tube 60 at a flange 136, forming a joint 132B therewith.

In an embodiment, a working length of the guidewire member 14 extends from about 100 to about 200 cm, usually from about 140 to about 180 cm, normally about 160 cm; with an external working diameter of the guidewire member ranging from about 0.007 to about 0.040 in., usually from about 0.009 to about 0.018 in., normally about 0.014 in.

In use, the access system can be delivered to the target site over a conventional guidewire. Once the access system has been positioned near the target site, the conventional guidewire can be removed and the elongate member (e.g., hollow guidewire) of the present invention can be advanced through an inner lumen of the access system to the target site. Optionally, the support system can be delivered concurrently with the advancement of the hollow guidewire. Alternatively, because the elongate member can have the flexibility, pushability, and torqueability to be advanced through the tortuous regions of the vasculature, the elongate member may be advanced through the vasculature to the target site without the use of the separate guidewire. In such embodiments, the access system can be advanced over the elongate member of the present invention to the target site. Once the elongate member has been positioned at the target site, the drive shaft is rotated, preferably, in an oscillation rotational mode, and advanced into the occlusive material or the entire elongate member may be advanced distally into the occlusion. The rotation of the drive shaft distal tip creates a path forward of the elongate member. In some embodiments, the path created by the distal tip has a path radius which is larger than the radius of the distal end of the elongate member. In other embodiments, the path created by the distal tip has a path radius which is the same size or smaller than the radius of the elongate member.

While not explicitly illustrated, a person of ordinary skill in the art will recognize that aspects of one configuration of the hollow guidewire body may be used with other configurations of the hollow guidewire body. Therefore, the above description should not be taken as limiting the scope of the invention which is defined by the appended claims.

What is claimed is:

1. A fixed deflection hollow device for crossing an occlusion or stenosis within a body lumen, the device comprising:
an elongate hollow body having a proximal end, a distal portion with a distal end having a pre-determined deflection as compared to a longitudinal axis of the hollow body, and an axial lumen extending between the proximal and distal ends; and
a mechanically movable core element extending through the axial lumen of the body.

2. A device according to claim 1, wherein the distal end of the hollow body is configured for maintaining the pre-determined deflection when it is radially unconstrained.

3. A device according to claim 2, wherein the hollow body is configured to be disposable within an inner passage of an access or therapeutic catheter with the deflected distal end being constrained within the inner passage while disposed therein.

4. A device according to claim 1, wherein an axially elongate member is fixedly disposed within a distal portion of the axial lumen of the elongate hollow body and applies tension which maintains the deflection of the hollow body.

5. A device according to claim 1, wherein the pre-determined fixed deflection is, at least in part, achieved by way of a shaped distal portion of the body.

6. A device according to claim 1, wherein the shaped distal end of the body is formed from a material having been pre-set to the pre-determined deflection angle.

7. A device according to claim 1, wherein the pre-determined deflection ranges from about 0 to about 60 degrees.

8. A device according to claim 1, wherein the pre-determined deflection ranges from about 5 to about 45 degrees.

9. A device according to claim 1, wherein the pre-determined deflection is about 15 degrees.

10. A device according to claim 1, wherein the core element comprises a drive shaft and a shaft distal tip extending distally from the distal end of the body and is configured for creating a passageway or enlarging an existing passageway through the occlusion or stenosis within the body lumen.

11. A device according to claim 10, wherein the core element is movable by way of rotational oscillation.

12. A device according to claim 1, wherein the core element is configured for rotational movement through an angle equal to or less than about 360 degrees.

13. A device according to claim 11, wherein the core element is configured for rotational movement induced by a motor which is configured for providing from about 100 to about 200,000 revolutions per minute.

14. A device according to claim 11, wherein the core element is configured for rotational movement induced by a motor which is configured for providing from about 5,000 to about 50,000 revolutions per minute.

15. A device according to claim 11, wherein the core element is configured for rotational movement induced by a motor which is configured for providing about 12,000 revolutions per minute.

16. A device according to claim 11, wherein the core element is configured for oscillation of the rotation angle in a time period ranging from about 0.2 to about 5.0 seconds.

17. A device according to claim 11, wherein the core element is configured for oscillation of the rotation angle in a time period ranging from about 0.3 to about 1.2 seconds.

18. A device according to claim 11, wherein the core element is configured for oscillation of the rotation angle in a time period of about 0.7 seconds.

19. A device according to claim 11, wherein a motor is configured to provide 140 cycles of rotations to the core element in about every 0.7 seconds.

20. A device according to claim 19, wherein the motor is configured to reverse its polarity of rotation after about 0.7 seconds.

21. A device according to claim 1, wherein the elongate hollow body comprises a plurality of sections.

22. A device according to claim 1, wherein the elongate hollow body comprises a unitary structure having a plurality of sections.

23. A device according to claim 21, wherein at least a section of the distal portion of the hollow body comprises an interrupted helical pattern.

24. A device according to claim 23, wherein the interrupted helical pattern comprises laser edged helical windings at 180 degrees interrupted by 30 degree segments.

25. A device according to claim 21, wherein at least a section of the distal portion of the hollow body comprises a ribbed pattern.

26. A device according to claim 21, wherein at least the distal portion of the body includes a first section with an interrupted helical pattern and a second section with a ribbed pattern and disposed distally from first section.

27. A device according to claim 10 further comprising an elongate tube extending along at least a longitudinal portion of the body and coupled to the distal portion thereof.

28. A device according to claim 27, wherein the elongate tube is distally tapered.

29. A device according to claim 27, wherein the elongate tube is formed from a material comprising nickel titanium alloy.

30. A device according to claim 10, further comprising a coil disposed over the distal portion of the drive shaft.

31. A device according to claim 30, wherein the coil disposed over the distal portion of the drive shaft is formed from radiopaque material.

32. A device according to claim 31, wherein the coil disposed over the distal portion of the drive shaft is formed from a platinum-iridium compound.

33. A device according to claim 27, wherein a polymeric insert is disposed about a proximal portion of the elongate tube and extends proximally to a handle assembly which is configured for mechanically operating the core element.

34. A device according to claim 1, wherein the drive shaft includes a distal extension extending beyond the drive shaft distal tip.

35. A device according to claim 27, wherein a coil is disposed over the distal portion of the drive shaft and radially separates the distal portion of the drive shaft from the distal portion of the elongate tube.

36. A device according to claim 35, wherein the coil disposed over the distal portion of the drive shaft is formed from a radiopaque material.

37. A device according to claim 27, wherein the elongate tube is fixedly connected to the distal end of the body.

38. A device according to claim 10, wherein the core distal tip is formed at least in part from a radiopaque material.

39. A device according to claim 26 further comprising a third section including a proximal coil extending proximally from the first section and is joined at a proximal end to a solid walled tube.

40. A device according to claim 26 further comprising a third section including a proximal coil extending proximally from the first section to the proximal end of the hollow body.

41. A device according to claim 39, wherein the first, second, and third sections together have a longitudinal dimension ranging from about 20 centimeters to about 60 centimeters.

42. A device according to claim 39, wherein the first, second, and third sections together have a longitudinal dimension of about 30 centimeters.

43. A device according to claim 26, wherein the first and the second sections together have a longitudinal dimension ranging from about 1 centimeter to about 5 centimeters.

44. A device according to claim 26, wherein the first and the second sections together have a longitudinal dimension of about 4 centimeters.

45. A device according to claim 10, wherein the drive shaft is distally tapered.

46. A device according to claim 21, wherein the distal portion comprises an interrupted helical patterned section extending proximally from the hollow body distal end and a coil section extending proximally from a proximal end of the interrupted helical patterned section.

47. A device according to claim 25, wherein the distal portion comprises a ribbed patterned section extending proximally from the hollow body distal end and a coil section extending proximally from a proximal end of the ribbed patterned section.

48. A device according to claim 26, wherein the first and second sections are independently formed from stainless steel, nickel-titanium, a radiopaque material, or a polymeric material.

49. A device according to claim 27, wherein the elongate tube is formed from a proximal section and a distal section longitudinally distanced from the proximal section and having proximal end disposed at the proximal end of the hollow distal portion.

50. A device according to claim 49, wherein the proximal and the distal sections are independently formed from a material selected from the group consisting of stainless steel, nitinol, and polymeric material.

51. A device according to claim 10, wherein at least a portion of the drive shaft is coated with a lubricious material.

52. A device according to claim 27, wherein the elongate tube is tapered at a proximal end and terminates at a solid tube forming the proximal portion of the hollow and forms a connection therewith.

53. A device according to claim 52, wherein the elongate tube terminates at a proximal end of the distal portion of the body and is fixedly attached thereat.

54. A device according to claim 52, wherein the elongate tube terminates at a distal end of the body.

55. A device according to claim 52, wherein a cuff is disposed about the elongate tube and the solid tube at the connection point.

56. A device according to claim 10, further comprising a handle assembly disposable at a proximal end of the hollow body for mechanically operating the core element.

57. A device according to claim 56, wherein the handle assembly is fixedly attachable to the proximal end of the hollow body and a proximal end of the core element.

58. A device according to claim 56, wherein the handle assembly is removably attachable to the distal end of the hollow body and a distal end of the core element.

59. A fixed deflection hollow device for crossing an occlusion or stenosis within a body lumen, the device comprising:
an elongate hollow body having a proximal end, a distal portion with a distal end having a pre-determined deflection as compared to a longitudinal axis of the hollow body, and an axial lumen extending between the proximal and distal ends; and
a mechanically movable core element movable by way of rotational oscillation extending through the axial lumen of the body, wherein the core element comprises a drive shaft and a shaft distal tip extending distally from the distal end of the body and is configured for creating a passageway or enlarging an existing passageway through the occlusion or stenosis within the body lumen.

60. A method of crossing an occlusion or stenosis within a body lumen, said method comprising:
positioning a distal end of a fixed deflection hollow device having an elongate hollow body having a proximal portion with a proximal end and a distal portion with the distal end which has a pre-determined deflection relative to the proximal portion adjacent to the occlusion or stenosis;
applying torque to the proximal end of the elongate hollow body to steer the deflected distal end of the elongate hollow body in the body lumen;
advancing the deflected distal end of the elongate member into the occlusion or stenosis; and
rotating and/or oscillating a core element having a drive shaft and distal tip disposed within an inner lumen of the hollow body with the core element distal tip extending distally beyond the distal end of the hollow body.

61. A method as in claim 60, wherein the occlusion or stenosis comprises a total occlusion.

62. A method as in claim 60, wherein the occlusion or stenosis comprises a chronic total occlusion.

63. A method as in claim 60, wherein the deflected distal end of the elongate hollow body is deflected by axial tension applied by an axially elongate member which is fixedly disposed within a distal portion of the axial lumen of the elongate hollow body.

64. A method as in claim 60, wherein the torque is applied by a handle assembly disposed at a proximal end of the guidewire device.

65. A method as claim 60, wherein the core member is rotated and/or oscillated by a motor in a handle assembly disposed at a proximal end of the hollow guidewire device.

66. A method as claim 65, wherein the core member is rotationally oscillated.

67. A method as in claim 60, wherein a handle assembly is removably disposable at a proximal end of the guidewire device.

68. A method as in claim 60, wherein a handle assembly is fixedly attached at a proximal end of the guidewire device.

69. A method as in claim 65, wherein the motor rotates at a rate of about 100 to about 200,000 revolutions per minute.

70. A method as in claim 65, wherein the motor rotates at a rate of about 5,000 to about 50,000 revolutions per minute.

71. A method as in claim 65, wherein the motor rotates at a rate of about 12,000 revolutions per minute.

72. A method as in claim 69, wherein the motor reverses its polarity in a time period ranging from about 0.2 to about 5.0 seconds.

73. A method as in claim 69, wherein the motor reverses its polarity in a time period ranging from about 0.3 to about 1.2 seconds.

74. A method as in claim 69, wherein the motor reverses its polarity in a time period of about 0.7 seconds.

75. A method as in claim 69, wherein the motor provides 140 cycles of rotation and changes the direction of rotation in about 0.7 seconds.

76. A method of crossing an occlusion or stenosis within a body lumen, said method comprising:
positioning a distal end of a guidewire adjacent to the occlusion or stenosis;
delivering an access system over the guidewire and positioning it adjacent to the occlusion or stenosis;
removing the guidewire while the access system is maintained in place;
advancing a distal end of a hollow device having an elongate hollow body having a proximal portion with a proximal end and a distal portion with the distal end which has a pre-determined deflection relative to the proximal portion through a lumen of the access system and positioning the deflected distal end of the hollow device adjacent to the occlusion or stenosis;
applying torque to the proximal end of the elongate hollow body to steer the deflected distal end of the elongate hollow body in the body lumen;
advancing the deflected distal end of the elongate member into the occlusion or stenosis; and
rotating and/or oscillating a core element having a drive shaft and distal tip disposed within an inner lumen of the hollow body with the core element distal tip extending distally beyond the distal end of the hollow body.
